(19) United States
(12) Patent Application Publication
(10) Pub. No.: US 2010/0167366 A1
(43) Pub. Date: Jul. 1, 2010

(54) PUMPING AND CONTAMINATION CONTROL SYSTEM FOR CELLULOSIC FEEDSTOCKS

(76) Inventor: DAVID A. STEWART, Boca Raton, FL (US)

Correspondence Address: FLEIT GIBBONS GUTMAN BONGINI & BIANCO P.L., ONE BOCA COMMERCE CENTER, 551 NORTHWEST 77TH STREET, SUITE 111, BOCA RATON, FL, 33487, US

(21) Appl. No.: 12/643,530

(22) Filed: Dec. 21, 2009

Related U.S. Application Data
(60) Provisional application No. 61/139,360, filed on Dec. 19, 2008.

(51) Int. Cl. C12P 7/06

(52) U.S. Cl. 435/161

(57) ABSTRACT

Processes for the pumping of various cellulosic feedstock materials that are used to produce an ethanol-containing beer are disclosed. In certain embodiments, the present invention relates to processes capable of pumping highly viscous slurries and/or feedstocks containing high levels of solids in an energy efficient manner, wherein the slurries of feedstocks are utilized in the production of ethanol.

### CROSS-REFERENCE TO RELATED APPLICATIONS

This application is based upon and claims priority from prior U.S. Provisional Patent Application No. 61/139,360, filed on Dec. 19, 2008, the entire disclosure of which is herein incorporated by reference. This Application is related to co-pending U.S. Provisional Patent Application No. 61/139,217 and U.S. Provisional Patent Application No. 61/139,268 each filed on Dec. 19, 2008, the entire disclosure of which each is herein incorporated by reference.

### FIELD OF THE INVENTION

This invention relates to processes for the pumping of various cellulosic feedstock materials that are used to produce ethanol. More particularly, the present invention relates to processes capable of pumping highly viscous slurries and/or feedstocks containing high levels of solids in an energy efficient manner, wherein the slurries of feedstocks are utilized in the production of ethanol.

### BACKGROUND OF THE INVENTION

Citrus waste consists primarily of peel, membranes and seeds, which result from processing citrus fruit for juice. Approximately 5 million tons of citrus waste are produced each year in Florida alone. Most of this peel waste is dried, pelletized, and sold as a beef or milk cattle feed filler known as citrus pulp pellets.

The global energy crisis, coupled with the effect fossil fuels are having on the environment, have led to continuing research in the area of alternative fuels. An attractive alternative is biomass fuels, such as ethanol. Ethanol produced from biomass is referred to as “cellulosic ethanol” and is usually defined as fuel ethanol produced from non-food crops such as agricultural residues (e.g., citrus peel waste (“CPW”), wheat straw, corn stover, bagasse, beet pulp, apple pommace, and corn husks), woody materials (e.g., hurricane debris, sawdust, soft wood, hard wood, and forestry waste), energy crops (e.g., switch grass, canes, and poplar trees) and waste materials like Municipal Solid Waste, MSW.

Among the citrus peel waste that has been studied is peel waste from Valencia oranges, the main citrus crop in Florida. The dry matter content observed for peel waste from Valencia oranges is reportedly 24-27% (Ting and Deszyck, 1961; Wilkins et al., 2005). Valencia peel having about 23% dry matter has been indicated to yield sugars for on a % dry matter basis (Grohmann and Baldwin, 1992; Grohmann et al., 1994, 1995) that theoretically provides ethanol in a yield of 6.6% by volume (5.2% by mass) (Grohmann et al., 1994).

A significant amount of research is being directed to producing ethanol from citrus waste. Citrus waste contains, among other things, several mono and disaccharides, mainly glucose, sucrose and fructose. Citrus waste also contains the polysaccharides cellulose, hemicellulose and pectin (Ting and Deszyck, 1961). Cellulose, hemicellulose and pectin can be hydrolyzed using a cocktail of pectinase, cellulase, and beta-glucosidase enzymes to produce glucose, fructose, arabinose, xylose, galactose, rhamnose, and galacturonic acid (GA) (Nishio and Nagai, 1979; Marshall et al., 1985; Ben-Shalom, 1986; Echeverria et al., 1988; Grohmann and Baldwin, 1992; Grohmann et al., 1994, 1995). Fructose, glucose, sucrose and galactose can be fermented by Saccharomyces cerevisiae yeast (typically used in the brewing industry) to produce ethanol (Grohmann et al., 1994).

The commercial recovery of alcohol by distillation from fermentation beers has been in widespread operation for many years. Control systems for improving quality within reasonable efficiency limits have paralleled the growth of this industry. However, in the energy context, rising costs and a need for greater efficiency has focused attention on the need for optimization of energy intensive (endothermic) processes through the application of dynamic control strategies. For example, the production of ethanol from grain blended with gasoline forms the motor fuel “gasohol.” To be effective as an alternative energy source, the process by which the ethanol is produced must minimize energy consumption so as to achieve “a net energy gain”.

There are a number of steps in the processing of cellulosic-based fermentation beers that may affect the “net energy gain,” particular on the front-end of the process. For example, a major limitation of known processes is the handling of the biomass feedstocks and their movement within the overall biomass to the ethanol production process train. Biomass feedstocks tend to be high in solids content, and/or potentially high in viscosity. These feedstocks are typically conveyed through the input and/or pretreatment stages of the bio-refinery by screw conveyor, belt conveyor, drag conveyor, or some equivalent system designed to handle high solids materials. Conveyor systems and/or their equivalents are typically expensive to install, require physical layouts which tend to be less flexible in design or operation, consume significant amounts of energy, and/or are plagued with high maintenance costs.

Operators have typically overcome these problems in previous pumping systems by diluting the biomass to make it suitable for pumping. However, this dilution is typically carried through the process and reduces ethanol concentrations in the fermented biomass. Diluting the feedstock so as to have the physical characteristics suitable for more traditional pumping systems would also, at a minimum, increases energy costs associated with later removal of water. However, it may ultimately result in an ethanol concentration which may be too dilute for economical purification, which reportedly requires an incoming ethanol concentration of at least 3%.

Further, in some situations, the source of the biomass may be some distance from the bio-refinery. The greater the distance between the biomass feedstock source and the bio-refinery, the more costly and less reliable conveyor systems tend to be relative to pumps. This is particularly an issue where the biomass is an agricultural residue or waste product being sourced from an operation that may be independent of, or located at, some distance within a plant from the bio-refinery.

Accordingly, there is considerable interest in developing new methods for the pumping of biomass feedstocks that are capable of handling typical viscosities and/or solids levels associated with these feedstocks. There is also interest in developing new methods for the efficient pumping of biomass feedstocks, and in some cases, without resorting to feedstock dilution to circumvent the higher viscosity and/or solids levels typically present. In other cases, there is interest in minimizing energy, capital and/or maintenance costs associated with pumping biomass feedstocks, especially in biomass to ethanol plant configurations. The present invention is directed to these and other important ends.

### SUMMARY OF THE INVENTION

Accordingly, it is an object of the present invention to provide methods for pumping biomass feedstocks.

It is another objective to minimize equipment capital costs and/or maintenance costs in moving the biomass feedstock.

It is another object to minimize energy use when moving the biomass feedstock.

It is another object to reduce, minimize, or prevent unwanted build up of contamination by microorganisms.

It is another object to utilize a re-circulating system for the diluting fluid that is used to reduce the viscosity or solids level in the biomass feedstock, thus reducing, minimizing, or preferably preventing the ultimate dilution of the fermented biomass.

It is another object to provide a partial solid-liquid separation using a screen, filter, centrifuge, mechanical press, or equivalent, prior to feeding the biomass feedstock into the pretreatment system to produce the diluting fluid that is used to reduce the viscosity or solids level in the biomass feedstock.

It is another object to include the use of an antimicrobial and or an antifungal substance to reduce, minimize, or preferably prevent contamination in the re-circulating fluid system due to the build-up of microorganisms.

It is another object to control pH to reduce, minimize, or preferably prevent contamination in the re-circulating fluid system due to the build-up of microorganisms.

It is another object to control temperature to reduce, minimize, or preferably prevent contamination in the re-circulating fluid system due to the build-up of microorganisms.

It is another object to use the waste heat available from the pre-treatment system, or the whole stillage dryer, or other available source of waste heat, to control the temperature to reduce, minimize, or preferably prevent contamination in the re-circulating fluid system due to the build-up of microorganisms.

It is another object to control residence time of the re-circulating system to reduce, minimize, or preferably prevent contamination in the re-circulating fluid system due to the build-up of microorganisms.

It is another object to efficiently transport the biomass material various distances between the source of the biomass and the pretreatment stage.

It is another object to effect a partial hydrolysis of the biomass within the pumping system by having enzymes or acids added to the mix/reaction tank that start the hydrolysis of the material to release fermentable sugars.

It is another object to prevent the loss of sugars, additives, and biomass material by re-circulating the fluid used to dilute the biomass feedstock.

It is another object to provide a pumping system for use in bio-refineries producing ethanol.

These and other objects will become apparent in the brief description of the drawings and the detailed description of the illustrative embodiments.

### BRIEF DESCRIPTION OF THE DRAWINGS

FIG. 1 is a simplified flow schematic diagram of a biomass feedstock pumping design of the present invention employing a re-circulating fluid loop for slurrying the biomass feedstock.

### DETAILED DESCRIPTION OF ILLUSTRATIVE EMBODIMENTS

The present invention is generally directed to processes for the pumping of various cellulosic feedstock materials, and in some embodiments, subsequently fermented to produce ethanol-containing beer and/or ethanol. More particularly, the present invention relates to processes capable of pumping highly viscous slurries and/or biomass feedstocks containing high levels of solids that may effectively transport biomass to downstream reactors capable of biomass conversion into ethanol in an energy efficient and economical manner.

The advantages of the methods of the present invention include without limitation adaptability to existing or cost-constrained plant configurations, reliability of the equipment used, and reduced impact on downstream operations, products, and by-products. For example, in certain embodiments, downstream product ethanol may be separated without excessive dilution with water (typically used to overcome issues associated with high viscosity and/or high solids levels). Another potential advantage, in certain applications, is that the whole stillage residue left after ethanol removal (which is typically dewatered into either animal feed or fuel for boiler systems) is not excessively diluted. Under such circumstances, any dewatering energy and financial costs are minimized.

As employed above and throughout the disclosure, the following terms, unless otherwise indicated, shall be understood to have the meanings indicated with the understanding that the examples provided are non-limiting.

As used herein, the terms “citrus” or “citrus fruit” includes all citrus fruits commercially available, including those in commercial production such as oranges, grapefruits, etc.

As used herein, the terms “citrus peel waste,” “citrus waste,” “citrus waste solids,” or “CPW” includes the citrus peel, segment membranes (pulp) and/or seeds.

As used herein, the term “biomass” refers to any renewable organic material used for the production of alternative fuels (such as ethanol) typically for its cellulose content rather than its starch or sugar content. Examples of biomass include, without limitation, citrus waste, wheat straw, corn stover, corn husks, rice straw, bagasse, beet pulp, pommace, woody materials (e.g., hurricane debris, sawdust, soft wood, hard wood, and forestry waste), energy crops (e.g., switch grass, canes, and poplar trees) and Municipal Solid Waste (“MSW”), whether used alone or in any combination. Such combinations include mixtures of biomass and crops typically of interest in ethanol fermentation primarily for their starch and/or sugar content, so long as the majority of fermentable sugars comes from biomass materials as defined herein. Crops such as potatoes, wheat, rye, triticale, corn, barley, sorghum and manioc and sugar cane juice, molasses, and beet sugars and the like are typically of interest in ethanol fermentation primarily for their starch and/or sugar content.

As used herein, the term “beer” refers to any ethanol containing mixture created by fermenting biomass, wherein biomass is as defined hereinabove.

As used herein, the terms “whole stillage” or “stillage” refers to the residue at the bottom of a still after fermentation, containing solids, but less alcohol compared to the beer prior to its distillation.

When ranges are used herein for physical properties, such as molecular weight, chemical properties or chemical formulae, all combinations and sub-combinations of ranges and specific embodiments therein are intended to be included.

The disclosures of each patent, patent application and publication cited or described herein are hereby incorporated by reference, in their entirety.

When any variable occurs more than one time in any constituent or in any process, its definition in each occurrence is independent of its definition at every other occurrence. Combinations of variables are permissible only if such combinations result in viable processes. When ranges are used for physical properties of components, or reaction conditions, such as weight percent, content, viscosity, temperatures, pressures, etc., all combinations and sub-combinations of ranges and specific embodiments therein are intended to be included.

It is believed that the names, characterizations, description, etc. used herein correctly and accurately reflect the chemicals, systems and methods. However, the nature and value of the present invention does not depend upon the theoretical correctness of these, in whole or in part. Thus, it is understood that the nomenclature is not intended to limit the invention in any way.

The present invention is directed, in part, to new processes for the pumping of biomass feedstocks, preferably where the biomass is a form of citrus waste.

In a first embodiment, the present invention provides processes for the pumping of biomass feedstocks, comprising:

(a) providing an aqueous slurry of a biomass feedstock;

(b) pumping the aqueous slurry from step (a) to a solid-liquid separator;

(c) separating at least a portion of the water from the aqueous slurry from step (b) to provide water and an increased solids biomass feedstock;

(d) employing the water separated in step (c) for preparing a subsequent aqueous slurry of biomass feedstock; and

(e) processing the increased solids biomass feedstock in a biomass to ethanol conversion plant.

In certain embodiments, the methods of the present invention reduce, minimize, or prevent unwanted build up of contamination by microorganisms.

In certain embodiments, the methods of the present invention utilize a re-circulating system for the diluting fluid that is used to reduce the viscosity or solids level in the biomass feedstock, thus reducing, minimizing, or preferably preventing the ultimate dilution of the fermented biomass.

In certain embodiments, the methods of the present invention provide a partial solid-liquid separation using a screen, filter, centrifuge, mechanical press, or equivalent, prior to feeding the biomass feedstock into the pretreatment system to produce the diluting fluid that is used to reduce the viscosity or solids level in the biomass feedstock.

In certain embodiments, the methods of the present invention include the use of an antimicrobial and or an antifungal substance to reduce, minimize, or preferably prevent contamination in the re-circulating fluid system due to the build-up of microorganisms.

In certain embodiments, the methods of the present invention control pH to reduce, minimize, or preferably prevent contamination in the re-circulating fluid system due to the build-up of microorganisms.

In certain embodiments, the methods of the present invention control temperature to reduce, minimize, or preferably prevent contamination in the re-circulating fluid system due to the build-up of microorganisms.

In certain embodiments, the methods of the present invention use the waste heat available from the pre-treatment system, or the whole stillage dryer, or other available source of waste heat, to reduce, minimize, or preferably prevent contamination in the re-circulating fluid system due to the build-up of microorganisms.

In certain embodiments, the methods of the present invention control residence time of the re-circulating system to reduce, minimize, or preferably prevent contamination in the re-circulating fluid system due to the build-up of microorganisms.

In certain embodiments, the methods of the present invention efficiently transport the biomass material potentially relatively long distances between the source of the biomass and the pretreatment stage.

In certain embodiments, the methods of the present invention effect a partial hydrolysis of the biomass within the pumping system by having enzymes or acids added to the mix/reaction tank in order to start the hydrolysis of the material to release fermentable sugars.

In certain embodiments, the methods of the present invention prevent the loss of sugars, additives, and biomass material by re-circulating the fluid used to dilute the biomass.

In certain embodiments, the methods of the present invention provide a pumping system for use in bio-refineries producing ethanol.

In certain embodiments, the methods of the present invention provide processes wherein the biomass feedstock comprises citrus waste.

In certain embodiments, the methods of the present invention provide processes wherein the initial ethanol-containing biomass mixture has a viscosity of from about 500 to about 20000 cP, preferably from about 1000 to about 10000 cP.

In certain embodiments, the methods of the present invention minimize energy use within the process, so as to greatly reduce the ethanol recovery cost and/or accomplish this without the need for expensive and/or burdensome membrane separation techniques.

In certain embodiments, the methods of the present invention involve reactions take all take place in vessels which are practical in construction and readily cleanable to prevent unwanted contamination by microorganisms.

In certain embodiments the biomass is provided to a mix/reaction tank, more preferably a mix/reaction tank designed to prevent stagnant or “dead spots”, still more preferably designed to help in contamination control. The tank optionally has a metal trap to reduce, minimize, or more preferably prevent metal objects from entering the downstream pump. Re-circulated water from previous liquid-solid separator operations is preferably added to the mix/reaction tank to achieve the desired viscosity and solids content for the downstream pumping system.

In certain embodiments, the pH in the mix/reaction tank is controlled, more preferably by the addition of base or acid substances.

In certain embodiments, the temperature in the mix/reaction tank is controlled, more preferably by the incoming temperature of the biomass and/or any re-circulating fluid from any aqueous waste heat source in the biomass to ethanol process train, still more preferably with water removed in the downstream liquid-separator operation. Additional temperature control is optionally provided via a heat exchanger or direct steam injection device, preferably a heat exchanger designed to recapture excess heat from a steam explosion step in the overall conversion process to ethanol from biomass.

In certain embodiments, de-watering aids, such as lime, are preferably added to the mix/reaction tank.

In certain embodiments, the pumping system effects a partial hydrolysis of the biomass feedstock by adding enzymes or acids to the mix/reaction tank to initiate the hydrolysis of the feedstock releasing at least a portion of the fermentable sugars therein.

In certain embodiments, the biomass slurry created in the mix/reaction tank is pumped, preferably with a positive displacement pump or equivalent to the input stage of the biomass pretreatment operation. In embodiments where the source of the biomass must be pumped a relatively long distance to the input stage of the biomass pretreatment operation additional pumping stages are optionally added.

In certain embodiments, a partial solid/liquid separation, preferably employing a screen, filter, centrifuge, mechanical press, or equivalent, is utilized between the mixing tank/reactor and the input stage of the biomass pretreatment operation, or its equivalent, to separate at least a portion of the water in the aqueous slurry. The separated water is further optionally preferably used as a re-circulating diluting fluid in providing the initial biomass feedstock slurry, more preferably; the recirculating fluid is first processed for control of microbial or fungal contamination prior to recirculation to the mixing tank/reactor.

In certain embodiments, the solids stream from the liquid-solids separator is processed for ethanol production. In preferred embodiments where the biomass is citrus waste, the re-circulating fluid is preferably pasteurized, more preferably by heating with heat from a heat exchanger that is used to condense the vapor stream from a steam stripping and or steam explosion pre-treatment operation. In certain embodiments, the condensed vapor streams contain citrus oil which may be decanted from the condensate and are preferably used as an antimicrobial and/or antifungal substance in a contamination control scheme for the re-circulating fluid.

In certain embodiments the re-circulating fluid that is returned to the mix/reaction tank is relatively rich in dissolved sugars and/or suspended biomass. In other embodiments, the re-circulating fluid contains contamination control additives and/or pre-hydrolysis additives, such as enzymes or acid(s). Recirculation of the fluid preferably minimizes the overall dilutive effect of the pumping system and/or maximizes additive performance and retention.

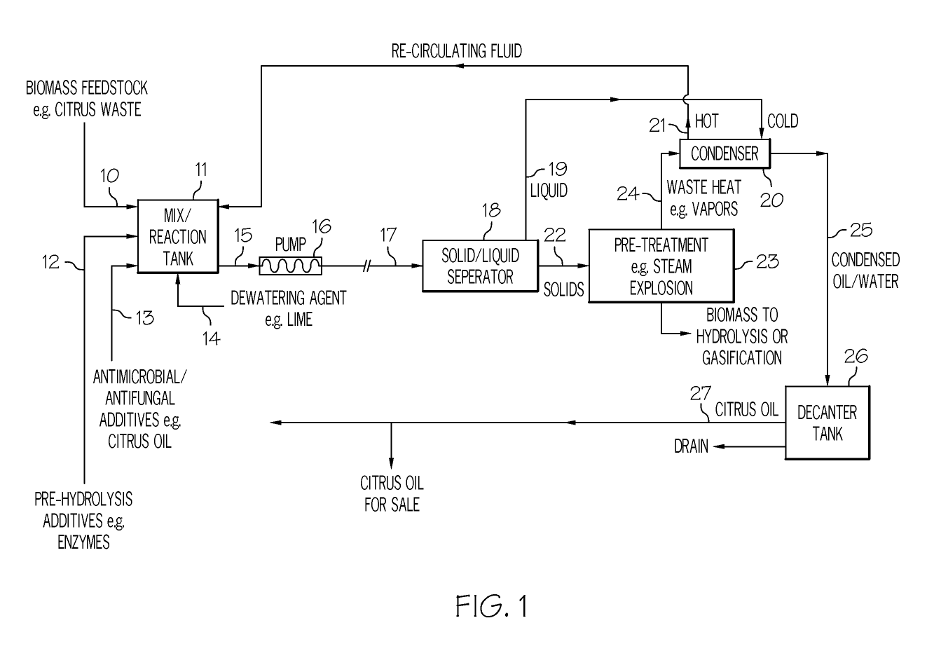

A simplified flow schematic design is shown in FIG. 1. FIG. 1 is not meant to limit the invention in any way, but rather, is used to illustrate certain aspects of the process. Biomass feedstocks such as citrus waste, wheat straw, corn stover, corn husks, bagasse, beet pulp, pommace, woody materials, energy crops and Municipal Solid Waste may be pumped alone or in any combination to provide the biomass for downstream fermentation in biomass beer.

The operation of the system shown in FIG. 1 is particularly advantageous where the feedstock has a high-solids content and/or high viscosity. In general, the level of solids and/or viscosity that the system may be efficiently handled is determined by pump selection. For example, current generation positive displacement pumps with auger feeds can operate with up to 40% dry solids content and viscosities up to 1,000,000 cP.

Referring to FIG. 1, the biomass feedstock 10, typically having a high-solids content and/or high viscosity, enters the mix/reaction tank 11 by a conveying device. Pre-hydrolysis additives 12, preferably additives such as acid or enzymes, may be added to tank 11, or at any other suitable point in the system. Care should be taken, in any pasteurization stage, so that in those processes where enzymes are added, the enzymes are not denatured, or the benefit of the pre-hydrolysis and liquefaction may not be maintained during re-circulation of the enzyme-containing diluting fluid.

Antimicrobial and/or antifungal substances 13 may be added to the mix/reaction tank 11. It may be preferable in some situations to utilize antimicrobial and or antifungal substances that may be naturally occurring within the feedstock to protect against process train, and particularly, recirculation fluid contamination.

In FIG. 1, citrus oil comprising limonene, is recovered at a later stage and may be added to the system at some point, such as the mix/reaction tank 11, to maintain a level in the range of 0.5% to 15%, with a preferred range of 2% to 5% as a naturally occurring antimicrobial or antifungal substance. The citrus oil is the fruit's natural defense against microorganisms and is typically found on the surface of the fruit. At an appropriate effective concentration, and preferably, with adequate mixing, the citrus oil or other additive may assist in the protection the entire biomass feedstock process stream.

Substance(s) 14, designed to help the solid/liquid separator function more efficiently, may be added to tank 11, or at any other suitable point in the system. In the schematic design shown, a base or other dewatering agent, preferably lime, may be added to increase the pH of citrus peel and/or help release water from the peel.

Water from the liquid-solid separation operation is re-circulated by addition to the mix tank to dilute the biomass feedstock. Once sufficient water has been added to the tank mixture, the slurry 15 is then dilute enough to be pumped 16. The limiting factor for the level of solids and/or viscosity that can be efficiently pumped is determined by pump selection. For example, current generation positive displacement pumps with auger feeds can operate with up to 40% dry solids content and viscosities up to 1,000,000 cP.

In the case of citrus peel with a 50% dilution factor, a suitable progressive cavity pump will be able to pump the biomass slurry for several hundred feet. If distances of several hundred yards are required then it may be necessary to have booster pump stages. At the end of the pipe run, the slurry 17 enters a partial solid/liquid separator 18.

The partial separation is achieved with a screen, mechanical press, centrifuge, filter, or equivalent. If a suitable level of separation can be achieved without mechanical energy, a screen may offer the lowest cost solution. The level of separation between the liquid and solid stream is determined by many factors including the level of dilution required for the pump 16. In the schematic design shown, a typical dilution would be 50%, therefore the volume of the liquid and solids streams are approximately equal. The feedstock characteristics will generally determine suitable dilution levels. By re-circulating the fluid from the partial solid/liquid separator 18 to dilute the biomass in the mixing tank/reactor 11, there is no significant loss of sugars, additives, or biomass material.

The liquid stream 19 may then be pumped, or gravity fed, directly back to the mix/reaction tank and the temperature within the mix/reaction tank 11 can be controlled via a heat exchanger or directly injected steam. In this scheme, the liquid stream 19 passes through a condenser, or heat exchanger, where the temperature of the liquid 21 as it leaves the unit 20 is suitable to pasteurize and, preferably in some embodiments, sterilize the liquid.

Typically the pasteurization temperature is determined by residence time, acceptable level of contamination, and suitable temperature levels for additives and the desired materials within the stream. For example, a residence time of several minutes would require a temperature of approximately 60° C., while a residence time of less than a minute would require a temperature of approximately 70° C. Alternatively, where an enzyme with a maximum operating temperature of 50° C. is employed in the pumping process, temperature in the heat exchanger must not exceed the denaturing temperature of the enzyme for any extended time period, or the enzyme's activity will be lost. When enzyme denaturation and contamination are issues in a particular process, it may be useful to rely on an additive, such as citrus oil, to control contamination.

The solids stream 22 may be conveyed, or potentially pumped over short distances, for processing by subsequent stages to complete the conversion into biofuel. Typically, this involves some type of pre-treatment stage 23 involving acid or enzymes if the material is to be hydrolyzed prior to, or simultaneously with, fermentation. The level of pre-hydrolysis that may be achieved within this pumping system varies from about a de minimus quantity to about quantitative levels of hydrolysis. While some low level of hydrolysis is more typical (for example, de minimus to about 30%, more preferably to about 20%, still more preferably to about 10% by weight of contained biomass feedstock), it may be possible with a suitable feedstock and a relatively rapid hydrolysis reaction to have the majority of the hydrolysis completed within the pumping system described here.

In some cases the solids stream 22 may be gasified and the syngas converted by a catalyst or microorganism to ethanol or other chemical.

In the schematic design shown, citrus waste is stripped of the fermentation inhibiting citrus oil by steam explosion. The vapor stream 24 from this stripping operation contains volatilized citrus oil, comprising limonene, preferably d-limonene, and steam. The vapor stream 24 enters a heat exchanger which condenses the vapor into a water and citrus oil mixture 25. The condensate 25 in the schematic design shown enters a decanter tank 26 where the citrus oil 27 is decanted off and is made available as an additive 13 or readied for sale or use as a valuable by-product.

A suitable additive 13 may not naturally occur in the biomass feedstock or an economic method of recovery may not be available, in which case any suitable commercially available alternative may be used.

Other features of the invention will become apparent in the course of the following exemplary embodiments that are given for illustration of the invention and are not to be construed as limiting the appended claims.

[Embodiment 1] A process for the pumping of biomass feedstocks, comprising:

(a) providing an aqueous slurry of a biomass feedstock;

(b) pumping the aqueous slurry from step (a) to a solid-liquid separator;

(c) separating at least a portion of the water from the aqueous slurry from step (b) to provide water and an increased solids biomass feedstock;

(d) employing the water separated in step (c) for preparing a subsequent aqueous slurry of biomass feedstock; and

(e) processing the increased solids biomass feedstock in a biomass to ethanol conversion plant.

[Embodiment 2] The process according to embodiment 1, wherein the biomass feedstock provided to step (a) comprises citrus waste.

[Embodiment 3] The process according to embodiment 1 or 2, wherein step (e) includes a steam explosion treatment step on the biomass feedstock.

[Embodiment 4] The process according to embodiment 1, 2, or 3, wherein an antimicrobial or antifungal additive is added to the aqueous slurry of step (a).

[Embodiment 5] The process according to embodiment 4, wherein the antimicrobial or antifungal additive is citrus oil.

[Embodiment 6] The process according to embodiment 1, 2, 3, or 4, wherein a cellulose hydrolytic enzyme added to the aqueous slurry of step (a).

[Embodiment 7] The process according to embodiment 1, 2, 3, 4, or 5, wherein lime is added to the aqueous slurry of step (a).

[Embodiment 8] The process according to embodiment 3, wherein waste heat from the steam explosion treatment step is removed by a heat exchanger.

[Embodiment 9] The process according to embodiment 8, wherein the water separated in step (c) is passed through the heat exchanger prior to the water's use in preparing a subsequent aqueous slurry of biomass feedstock.

[Embodiment 10] The process according to embodiment 1, 2, 3, 4, 5, 6, 7, 8, or 9, wherein the process is as described in FIG. 1.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A process for the pumping of biomass feedstocks, comprising:
(a) providing an aqueous slurry of a biomass feedstock;
(b) pumping the aqueous slurry from step (a) to a solid-liquid separator;
(c) separating at least a portion of the water from the aqueous slurry from step (b) to provide water and an increased solids biomass feedstock;
(d) employing the water separated in step (c) for preparing a subsequent aqueous slurry of biomass feedstock; and
(e) processing the increased solids biomass feedstock in a biomass to ethanol conversion plant.

2. The process according to claim 1, wherein the biomass feedstock provided to step (a) comprises citrus waste.

3. The process according to claim 1, wherein step (e) includes a steam explosion treatment step on the biomass feedstock.

4. The process according to claim 1, wherein an antimicrobial or antifungal additive is added to the aqueous slurry of step (a).

5. The process according to claim 4, wherein the antimicrobial or antifungal additive is citrus oil.

6. The process according to claim 1, wherein a cellulose hydrolytic enzyme added to the aqueous slurry of step (a).

7. The process according to claim 1, wherein lime is added to the aqueous slurry of step (a).

8. The process according to claim 3, wherein waste heat from the steam explosion treatment step is removed by a heat exchanger.

9. The process according to claim 8, wherein the water separated in step (c) is passed through the heat exchanger prior to the water's use in preparing a subsequent aqueous slurry of biomass feedstock.

10. The process according to claim 1, wherein the process is as described in FIG. 1.
